(19) United States
(12) Patent Application Publication
(10) Pub. No.: US 2025/0381310 A1
(43) Pub. Date: Dec. 18, 2025

(54) NIR ABSORBING CAPSULES

(71) Applicant: AGFA-GEVAERT NV, Mortsel (BE)

(72) Inventors: Johan Loccufier, Mortsel (BE); Jos Louwet, Mortsel (BE)

(73) Assignee: AGFA-GEVAERT NV, Mortsel (BE)

(21) Appl. No.: 18/284,749

(22) PCT Filed: Mar. 28, 2022
(86) PCT No.: PCT/EP2022/058063

§ 371 Date: Sep. 28, 2023

(30) Foreign Application Priority Data
Apr. 1, 2021 (EP) 21166542.7

(51) Int. Cl. A61K 49/00; A61K 41/00; A61K 47/34

(52) U.S. Cl. CPC A61K 49/0091; A61K 41/0052; A61K 41/0057; A61K 47/34; A61K 49/0032

(57) ABSTRACT

A capsule comprising a polymeric shell surrounding a NIR absorber, the polymeric shell comprises a poly(amino acid) and is obtainable by interfacial polymerization of a N-carboxy-anhydride monomer according to general structure (I). The capsule is suitable for opto-medical applications such as phototherapies including photothermal therapy (PTT), photodynamic therapy (PDT), photo stimulated drug release and fluorescence medical imaging.

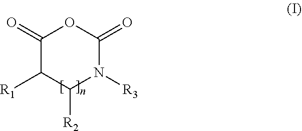

### TECHNICAL FIELD

The present invention relates to a biocompatible organic nano- and microcapsule design for opto-medical applications such as phototherapies including photothermal therapy (PTT), photodynamic therapy (PDT), photo stimulated drug release and fluorescence medical imaging

### BACKGROUND ART

Near infrared (NIR) laser technology is gaining importance in non-invasive treatment of different diseases and in medical diagnostics, including photothermal therapy, photodynamic therapy, fluorescence imaging and photo-acoustic imaging. Several of these technologies rely on NIR absorbing nanoparticles, where often inorganic nanoparticles are used, such as gold nanomaterials, carbon nanomaterials, including carbon nanotubes, metal sulfides, metal oxides and different upconverting nanoparticles. The use of inorganic optothermal converting nanoparticles has extensively been reviewed (Raza et al., Journal of Materials Research and Technology, 8(1), 1497-1509 (2019); Wang et al., International Journal of Nanomedicine, 15, 1903-1914 (2020)). Although giving excellent NIR response, these nanoparticles are not biodegradable and hold the risk for bioaccumulation and long retention time in the body that could potentially increase their probability of long term toxicity. Therefore, nanoparticles based on biocompatible organic NIR absorbers would be highly preferred.

Although several classical organic NIR absorbers are well documented in opto-medical applications, cyanine dyes are a particularly preferred class of NIR absorbers due to their high molar extinction coefficient. High molar extinction of NIR laser light has the advantage that the required amount of NIR absorber can be decreased and makes application of in vitro and in vivo imaging and treatment of deeply-sited diseases such as tumours, possible. One of the best known and also FDA approved cyanine dye, is indocyanine green.

NIR absorbers such as indocyanine green have to be encapsulated in to increase their lifetime in the body, be dispersible in the aqueous fluids of the body, prevent photo-bleaching and increase its tumour targeting ability.

Encapsulating NIR absorbers have furthermore other additional benefits. It is known that the spectral characteristics of several classes of NIR absorbers are very dependent on their environment and can change in function of e.g. pH and ionic strength, caused by aggregation phenomena. Therefore, physically encapsulating NIR absorbers makes the spectral characteristic and photophysics of the absorber independent of the external environment. The response in a physiological environment becomes then very predictable. Tuning the laser response is simply done by adapting the concentration of the NIR absorber in the capsule, avoiding laborious synthesis of NIR absorbers each time.

Polypeptide-based materials such as poly(amino acids) are valid candidates for encapsulation due to their biocompatibility, biodegradability, high chemical functionality, tunable structural architecture and ability to form nano- or microcapsules.

Encapsulation of NIR absorbers by means of poly(amino acids) is commonly achieved via coacervation or formation of micelles. The poly(amino acids) are prepared by the ring-opening polymerization of N-carboxy-anhydride monomers (NCA's).

In order to form micelles, amphiphilic block copolymers containing poly(amino acid) blocks have to be prepared separately and assembled into micelle like capsules or transferred into capsules using coacervation type of approaches. The self-assembly of amphiphilic block copolymers into micelles can hold up NIR absorbers.

In another approach, coacervation is achieved by combining anionic poly(amino acid) electrolytes together with cationic poly(amino acid) electrolytes as disclosed in ACS Macro Lett. 2014, 3, 1088-1091 and in Chem. Lett. 2012, 41, 13541356. Coacervation always requires at least two polyelectrolytes, hence limiting the choice of useful poly(amino acids). The shell of the obtained capsule is hold together by electrostatic forces between the polyelectrolytes and is susceptible to water penetration, hence leading to a substantive water permeability towards the core of the capsule and hence towards the encapsulated compound(s).

Both technologies deliver capsules or micelles having the disadvantage of a much weaker shell than a capsule with a polymeric shell. In many systems, a crosslinking of the shell of micellar systems is then required to assure bio-stability.

Poly(amino acids) can be prepared by the polymerization of N-carboxy-anhydride monomers (NCA's) in a heterogeneous water-solvent-system. Wang et al. (International Journal of Biological Macromolecules Elsevier BV, NL, Volume 42, No. 1, p. 450-454) described the preparation of glycopeptide microspheres starting from acylated chitosan as initiator for graft-polymerization of NCA's in a heterogeneous water-solvent mixture. The emulsification of the aqueous phase into the solvent phase was particularly critical only allowing the formation of larger particles in the order of magnitude of 100 micron to 800 micron with shell thicknesses of around 50 micron. The particle size is completely out of range for a lot of applications, including several biomedical applications, where particle sizes well below 1 micron are needed. The disclosed microspheres were prepared using L-leucine as amino acid and did not contain specific core material. Translating the disclosed method to an oil in water methodology, which is by far preferred, as it does not require full evaporation followed by redispersing in water, is far from obvious. In the proposed methodology, the compound to be encapsulated has to be water soluble, as water is the discontinuous phase. The method does not allow to encapsulate more hydrophobic compounds in a single step, which only can be introduced by reloading the isolated capsules, making this type of encapsulation very laborious and economically not feasible for a lot of applications

Biocompatible capsules or micelles for use in photo thermal therapy, photodynamic therapy, photo stimulated drug delivery and fluorescence medical imaging, need to have stealth properties to avoid uptake by the reticuloendothelial system and only act or release drug at the required site in a controlled manner. Stealth properties can be introduced into carriers such as capsules and micelles through incorporation of synthetic polymers with inherent stealth properties such as poly(ethylene glycol) (PEG). Incorporation of PEG often requires laborious synthetic protocols of the polyelectrolytes or amphiphilic block copolymers prior to encapsulation, hampering easy and scalable preparation of NIR absorbing poly(amino acid) based capsules.

Micelle like capsules mostly need a liquid medium to retain its spherical structure such as to hold the core material in the inside of the micelle. Isolation of the micelle in a dried state is hence very difficult or not possible. Micelles and capsules obtained via coacervation have a limited range of obtainable particle size in contrast to capsules obtained by interfacial polymerization. Furthermore, the approaches by means of amphiphilic block copolymers allow very good control on the polymer structure but require exhaustive synthetic procedures to prepare the well-defined polymers, making them less suitable for technical applications in contrast to interfacial polymerization based technologies.

Nano- and microcapsules can be prepared using both chemical and physical methods. For technological applications, interfacial polymerisation is a particularly preferred industrial technology as they allow the highest control in designing the capsules.

WO2018/234179 discloses capsules prepared via interfacial polymerisation and which comprise a shell of vinylogous-urethane, vinylogous-amide or vinylogous-urea units and a core which may comprise reactive chemistry in combination with IR absorbing dyes. Shells of vinylogous-urethane, vinylogous-amide or vinylogous-urea do not show a biodegradability.

Therefore, there is a need for aqueous based single step encapsulation technologies, allowing direct access to aqueous dispersions of poly(amino acid) based capsules, encapsulating a wide range of compounds over a broad scope of particle sizes, including submicron particle sizes, comprising a shell showing a high mechanical strength, a low water permeability, and which shell is biodegradable.

### SUMMARY OF INVENTION

It is an object of the invention to provide solution to the above stated problems. The solution is realized by means of NIR absorbers encapsulated with poly(amino acids) by means of an industrial and easy scalable technology as defined in Claim 1.

It is a further aspect of the present invention to provide an aqueous dispersion of the capsules as defined in Claim 1. The aqueous dispersion is defined in Claim 10.

According to another aspect, the present invention includes an industrial scalable method of encapsulating NIR absorbers with poly(amino acids) as defined in Claim 13.

Other features, elements, steps, characteristics and advantages of the present invention will become more apparent from the following detailed description of preferred embodiments of the present invention. Specific embodiments of the invention are also defined in the dependent claims.

### DESCRIPTION OF EMBODIMENTS

### A. The Capsule

The objects of the present invention are realized by a capsule, wherein the core comprises a NIR absorber and the shell comprises an oligo- or poly(amino acid), obtained by oligomerization or polymerization of at least one N-carboxy-anhydride monomer according to general formula I

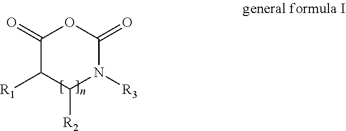

wherein
n represents 0 or 1
R₁, R₂ and R₃ are selected from the group consisting of a hydrogen, a substituted or unsubstituted alkyl group, a substituted or unsubstituted alkenyl group, a substituted or unsubstituted alkynyl group, a substituted or unsubstituted aralkyl group, a substituted or unsubstituted alkaryl group and a substituted or unsubstituted aryl or heteroaryl group

Any of R₁, R₂ and R₃ may represent the necessary atoms to form a five to eight membered ring.

The particle size of the capsules of the invention is preferably from 0.05 μm to 10 μm, more preferably from 0.07 μm to 5 μm and most preferably from 0.1 μm to 3 μm. Capsules according to the present invention having a particle size below 1 μm are particularly preferred as they reduce the risk of capillary clogging in administering needles and tubes and in preventing phagocytosis.

### A.1. The N-Carboxy-anhydryde Monomer

The objects of the present invention are realized by a capsule obtainable by oligomerization or polymerization of at least one N-carboxy-anhydride monomer according to general structure I.

In a preferred embodiment n represents 0. In a particular preferred embodiment R₃ represents a hydrogen or an alkyl group, a hydrogen being the most preferred.

In another preferred embodiment R₁ and R₂ are selected from the group consisting of a hydrogen, a substituted or unsubstituted alkyl group, a substituted or unsubstituted aralkyl group, a substituted or unsubstituted alkaryl group and a substituted or unsubstituted aryl group.

In further preferred embodiment, the N-carboxy-anhydride monomer according to general structure is selected from the group consisting of a glycine derivative, an alanine derivative, a leucine derivative, a phenylalanine derivative, a phenylglycine derivative, a valine derivative, a glutamic acid derivative, an aspartic acid derivative, a lysine derivative, an ornithine derivative, a histidine derivative, a methionine derivative, a cysteine derivative, an arginine derivative, a tryptophane derivative, a cysteine derivative, an isoleucine derivative, a tyrosine derivative, a proline derivative and a serine derivative. Both D- and L-amino acid derivatives and mixtures thereof can be used.

Typical N-carboxy-anhydride monomers are given in Table 1 without being limited thereto.

TABLE 1

| | |
|---|---|
| 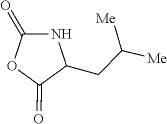 [SMILES: CC(C)CC1NC(=O)OC1=O] | NCA-1 |
| 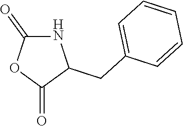 [SMILES: O=C1NC(Cc2ccccc2)C(=O)O1] | NCA-2 |
| 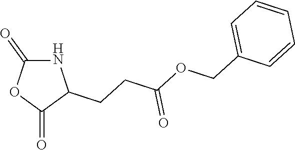 [SMILES: O=C(CCC1NC(=O)OC1=O)OCc1ccccc1] | NCA-3 |
| 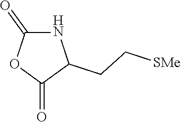 [SMILES: CSCCC1NC(=O)OC1=O] | NCA-4 |
| 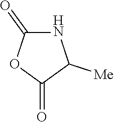 | NCA-5 |
| 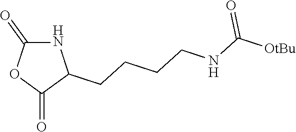 [SMILES: CC(C)(C)OC(=O)NCCCCC1NC(=O)OC1=O] | NCA-6 |
| 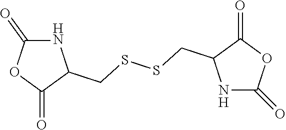 [SMILES: O=C1NC(CSSCC2NC(=O)OC2=O)C(=O)O1] | NCA-7 |
| 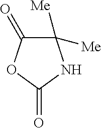 | NC-8 |
| 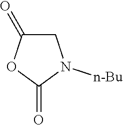 | NCA-9 |
| 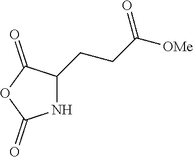 [SMILES: COC(=O)CCC1NC(=O)OC1=O] | NCA-10 |
| 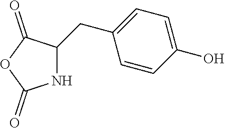 [SMILES: O=C1NC(Cc2ccc(O)cc2)C(=O)O1] | NCA-11 |
| 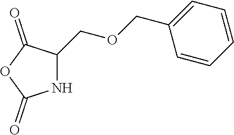 [SMILES: O=C1NC(COCc2ccccc2)C(=O)O1] | NCA-12 |

N-carboxy-anhydrides (NCA's) have been prepared using different synthetic methodologies, starting with the oldest method, known as Leuchs' method, starting from chloroformate acylation of the amino acid, followed by conversion to the corresponding NCA via its acid chloride. Several variants have been published on this methods, by Wessely and by Katchalski, respectively using a mixed anhydride method and a conversion using PBr₃. Probably, the most well-known method is the Fuchs-Farting method, using phosgene for direct conversion of the amino acid to the corresponding NCA. For safety reasons, phosgene has been replaced by di- or triphosgene in later research. Over the last years, several phosgene free methodologies have been disclosed. The methodologies have been reviewed by Secker et al. (Macromol. Biosci., 15, 881-891 (2015)).

### A.2. The NIR Absorber

Any organic near infrared (NIR) absorber known in the art can be used in the current invention, with the proviso that the NIR absorber is soluble in at least one water immiscible solvent. A water immiscible solvent is defined as a solvent that forms a two phase system at room temperature when mixed with water in a one to one ratio. Esters and ketones are particularly preferred water immiscible solvents.

Typical NIR absorbers can be selected from the group consisting of polymethyl indoliums, metal complex IR dyes, indocyanine green, polymethine dyes, croconium dyes, cyanine dyes, merocyanine dyes, squarylium dyes, chalcogenopyryloarylidene dyes, metal thiolate complex dyes, bis(chalcogenopyrylo)polymethine dyes, oxyindolizine dyes, bis(aminoaryl)polymethine dyes, indolizine dyes, pyrylium dyes, quinoid dyes, quinone dyes, phthalocyanine dyes, naphthalocyanine dyes, azo dyes, (metalized) azomethine dyes and combinations thereof.

Cyanine dyes are a particularly preferred class of NIR absorbers, due to their high extinction coefficient.

Cyanine dyes showing a high solubility in organic solvents are particularly preferred as they are easily incorporated in the core of the capsules of the invention by means of interfacial polymerisation. The cyanine dye according to general formula II is therefore particularly preferred for designing a nanoparticle according to the present invention.

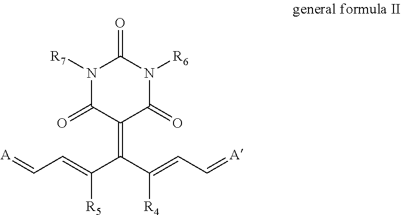

wherein
A and A′ independently represent a substituted or unsubstituted heterocyclic group, covalently bonded to the polymethine chromophore via a carbon atom
R₄ and R₅ are independently selected from the group consisting of a hydrogen, a substituted or unsubstituted alkyl group, a substituted or unsubstituted alkenyl group, a substituted or unsubstituted alkynyl group, a substituted or unsubstituted aralkyl group, a substituted or unsubstituted alkaryl group and a substituted or unsubstituted aryl or heteroaryl group R₄ and R₅ may represent the necessary atoms to form a five to eight membered ring
R₆ and R₇ are independently selected from the group consisting of a substituted or unsubstituted alkyl group, a substituted or unsubstituted alkenyl group, a substituted or unsubstituted alkynyl group, a substituted or unsubstituted aralkyl group, a substituted or unsubstituted alkaryl group and a substituted or unsubstituted aryl or heteroaryl group.

In a further preferred embodiment, said NIR absorber represents a compound according to general formula III:

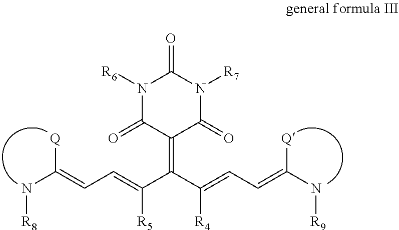

wherein,
R₄ and R₅ are independently selected from the group consisting of a hydrogen, a substituted or unsubstituted alkyl group, a substituted or unsubstituted alkenyl group, a substituted or unsubstituted alkynyl group, a substituted or unsubstituted aralkyl group, a substituted or unsubstituted alkaryl group and a substituted or unsubstituted aryl or heteroaryl group R₄ and R₅ may represent the necessary atoms to form a five to eight membered ring
R₆ and R₇ are independently selected from the group consisting of a substituted or unsubstituted alkyl group, a substituted or unsubstituted alkenyl group, a substituted or unsubstituted alkynyl group, a substituted or unsubstituted aralkyl group, a substituted or unsubstituted alkaryl group and a substituted or unsubstituted aryl or heteroaryl group
R₈ and R₉ independently represent a substituted or unsubstituted alkyl group, a substituted or unsubstituted alkenyl group, a substituted or unsubstituted alkynyl group, a substituted or unsubstituted aralkyl group, a substituted or unsubstituted alkaryl group and a substituted or unsubstituted aryl or heteroaryl group
Q represents the necessary atoms to form a substituted or unsubstituted five or six membered heteroring

In a further preferred embodiment, R₄ and R₅ represent the necessary atoms to form a substituted or unsubstituted five or six membered ring, a five membered ring being the most preferred.

In another preferred embodiment, R₆ and R₇ independently represent a substituted or unsubstituted alkyl group, a substituted or unsubstituted alkenyl group, a substituted or unsubstituted alkynyl group and a substituted or unsubstituted aralkyl group, a substituted or unsubstituted alkyl group being more preferred.

In a further preferred embodiment, A and A′ independently are selected from the group consisting of a substituted or unsubstituted indolinine, a substituted or unsubstituted naphtinolinine, a substituted or unsubstituted naphtostyryl group, a substituted or unsubstituted benzimidazole, a substituted or unsubstituted benzothiazole, a substituted or unsubstituted benzoxazole, a substituted or unsubstituted pyridine and a substituted or unsubstituted quinoline. Indolinines, naphtindolinines and naphtostyryls are particularly preferred.

In an even further preferred embodiment, at least one and more preferably at least two of R₆, R₇, R₈ and R₉ represent a branched substituted or unsubstituted alkyl group.

A branched alkyl group is defined as an alkyl group wherein at least a second group selected from the group consisting of an alkyl group, an alkenyl group, an alkynyl group, an aralkyl group, an alkaryl group and an aryl or heteroaryl group is substituted on a non-terminal carbon atom of the alkyl chain. Most preferably, said branched alkyl group is substituted by an alkyl group.

Typical examples of NIR absorber according to the present invention are given in Table 2 without being limited thereto.

TABLE 2

| | |
|---|---|
| 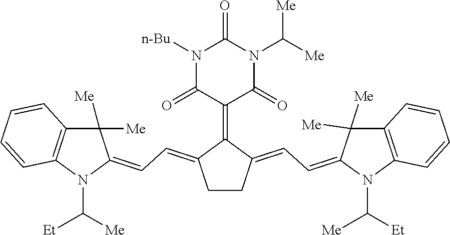 | NIR-1 |
| 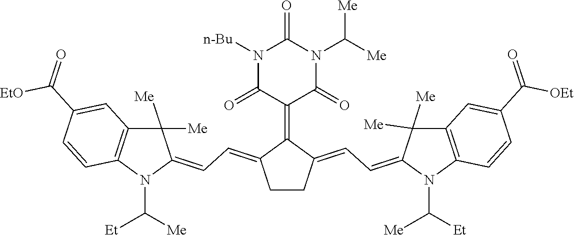 | NIR-2 |
| 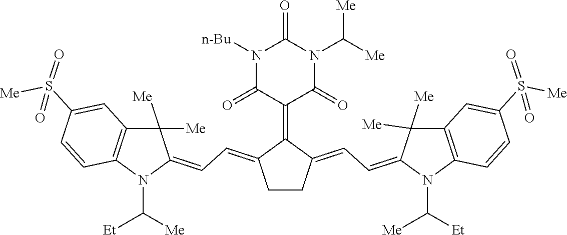 | NIR-3 |
| 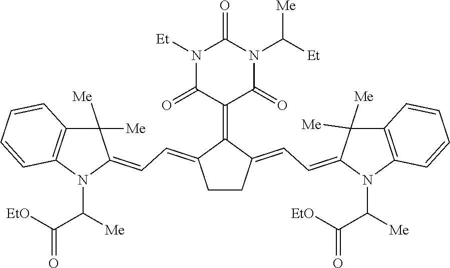 [SMILES: CCOC(=O)C(C)N1/C(=C/C=C2\CC/C(=C\C=C3\N(C(C)C(=O)OCC)c4ccccc4C3(C)C)C2=C2C(=O)N(CC)C(=O)N(C(C)CC)C2=O)C(C)(C)c2ccccc21] | NIR-4 |
| 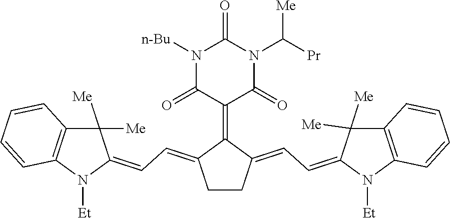 | NIR-5 |
| 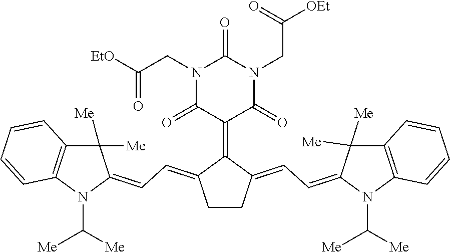 [SMILES: CCOC(=O)CN1C(=O)C(=C2/C(=C/C=C3/N(C(C)C)c4ccccc4C3(C)C)CC/C2=C\C=C2\N(C(C)C)c3ccccc3C2(C)C)C(=O)N(CC(=O)OCC)C1=O] | NIR-6 |
| 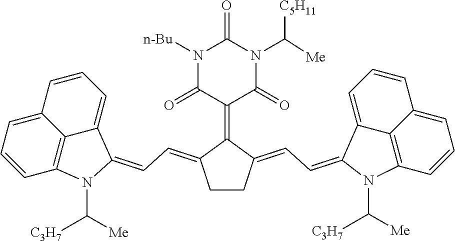 | NIR-7 |
| 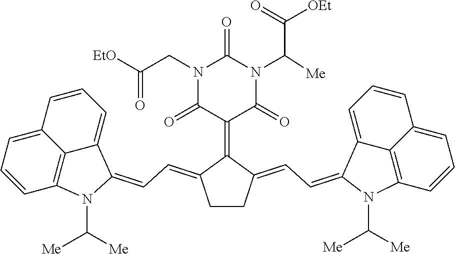 [SMILES: CCOC(=O)CN1C(=O)C(=C2/C(=C/C=c3\c4cccc5cccc(c54)n3C(C)C)CC/C2=C\C=c2/c3cccc4cccc(c43)n2C(C)C)C(=O)N(C(C)C(=O)OCC)C1=O] | NIR-8 |
| 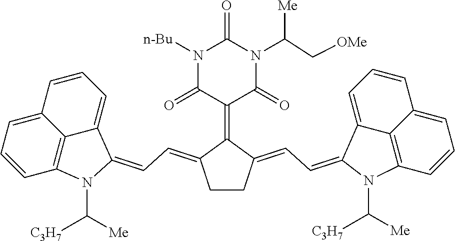 | NIR-9 |
| 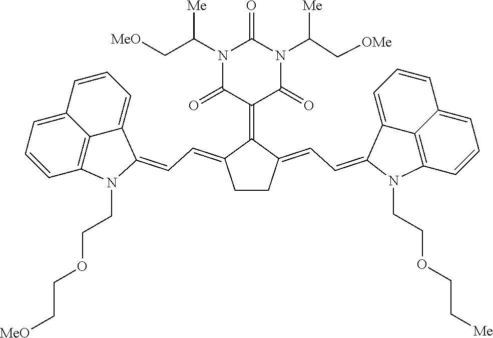 [SMILES: CCCOCCn1/c(=C/C=C2\CC/C(=C\C=c3/c4cccc5cccc(c54)n3CCOCCOC)C2=C2C(=O)N(C(C)COC)C(=O)N(C(C)COC)C2=O)c2cccc3cccc1c32] | NIR-10 |
| 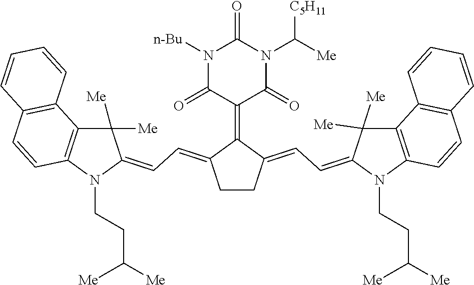 | NIR-11 |
| 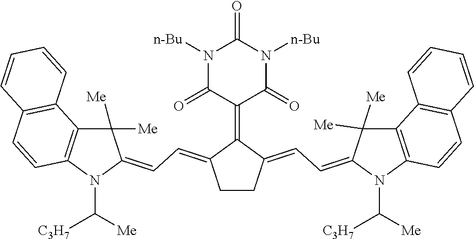 | NIR-12 |
| 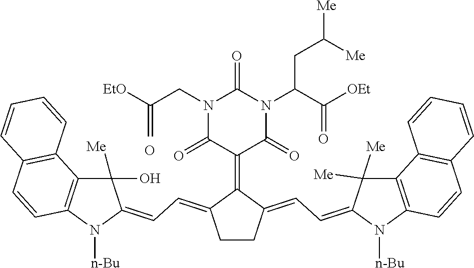 | NIR-13 |
| 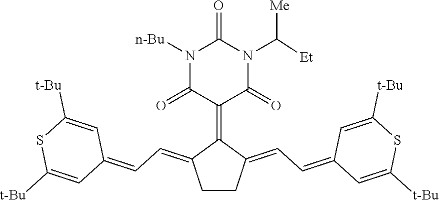 | NIR-14 |
| 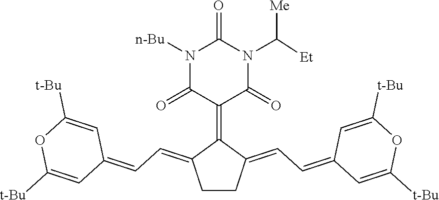 | NIR-15 |
| 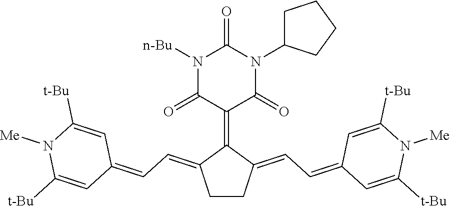 | NIR-16 |
| 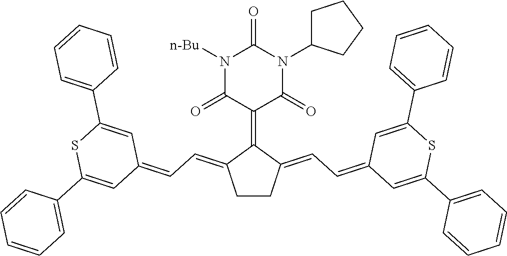 | NIR-17 |
| 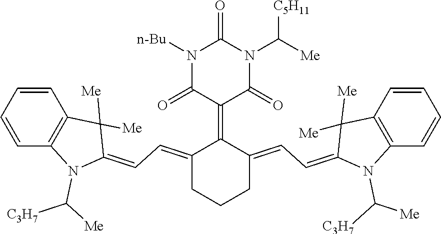 | NIR-18 |
| 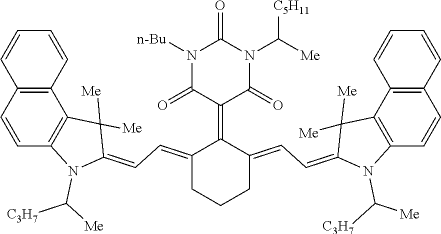 | NIR-19 |
| 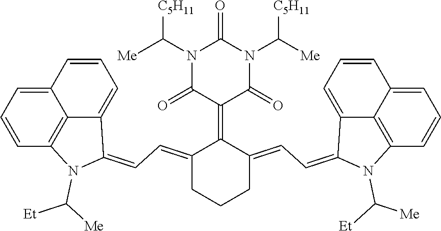 | NIR-20 |
| 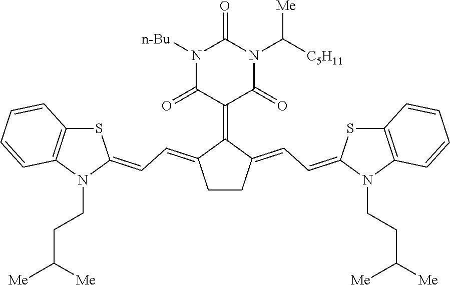 | NIR-21 |
| 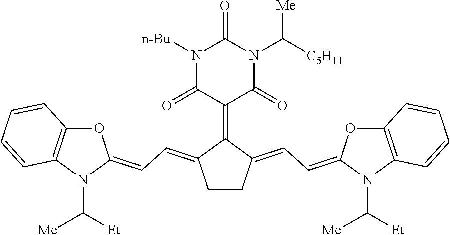 | NIR-22 |
| 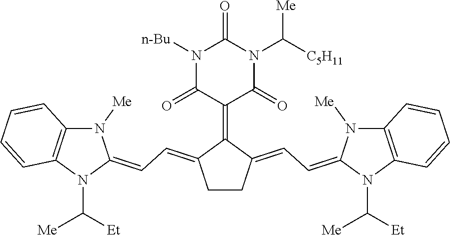 | NIR-23 |
| 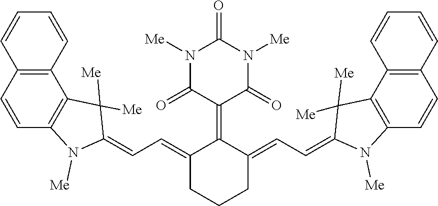 [SMILES: CN1C(=O)C(=C2/C(=C\C=C3\N(C)c4ccc5ccccc5c4C3(C)C)CCC/C2=C\C=C2\N(C)c3ccc4ccccc4c3C2(C)C)C(=O)N(C)C1=O] | NIR-24 |
| 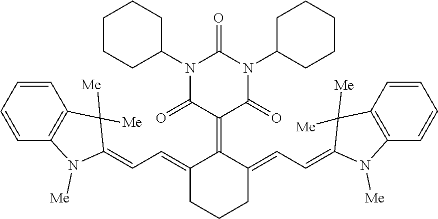 [SMILES: CN1/C(=C/C=C2/CCC/C(=C\C=C3\N(C)c4ccccc4C3(C)C)C2=C2C(=O)N(C3CCCCC3)C(=O)N(C3CCCCC3)C2=O)C(C)(C)c2ccccc21] | NIR-25 |
| 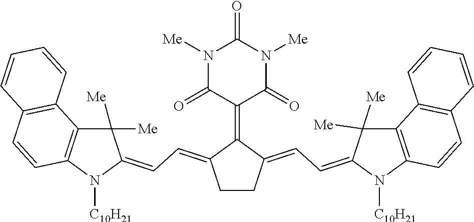 | NIR-26 |
| 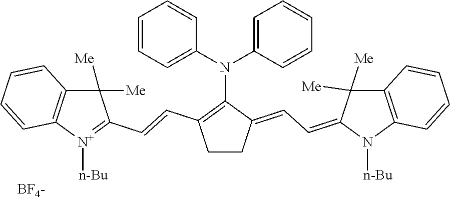 | NIR-27 |
| 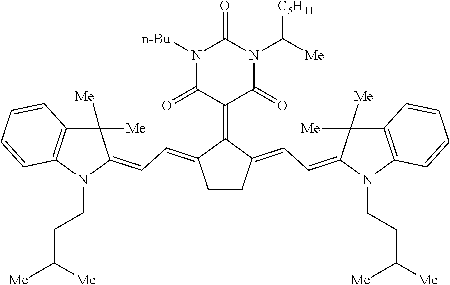 | NIR-28 |

The NIR absorber preferably has an absorption maximum between 700 and 1200 nm, more preferably between 750 and 1150 nm and most preferably between 780 and 1100 nm.

The NIR absorber content in the dispersion is preferably between 0.05 wt. % to 15 wt. % on the total solid content of the dispersion, more preferably between 0.1 wt. % and 10 wt. % and most preferably between 0.25 wt. % and 5 wt. %.

### A.3. Pharmaceutical Active Compound

The capsules of the invention are also suitable for on-demand drug release wherein the drug is released upon heating the particles by means of an appropriate NIR light source such as a NIR laser. Therefor it is useful to incorporate a pharmaceutical compound to achieve this on-demand drug release.

Sometimes, PTT or PDT cannot completely destruct cancer cells and may result in the survival of the residual cells after photothermal treatment. Therefor it is useful to incorporate anti-cancer drugs for enhanced chemotherapy. The drug will be released upon application of NIR light on the composite particle due to the heat generated, triggering synergetic chemo-photothermal therapy. The anti-cancer drug should preferably be soluble in the water immiscible solvent used in the preparation of the composite resin particles (see § A.4.).

Anti-cancer drugs which are suitable to be incorporated in the particles of the invention are cytostatics. Cytostatics for the treatment of cancer can be selected from the group consisting of alkylating agents, anthracyclines, cytoskeletal disruptors, epothilones, histone deacetylase inhibitors, topoisomerase I inhibitors, topoisomerase II inhibitors, kinase inhibitors, nucleotide analogs, peptide antibiotics, platinum based agents, retinoids and vinca alkaloids and derivatives. Alkylating agents can be bi- or monofunctional. Typical bifunctional alkylating agents are cyclophosphamide, mechlorethamine, chlorambucil and melphalan. Typical monofunctional alkylating agents are dacarbazine, nitrosoureas and temozolomide. Typical anthracyclines are daunorubicin, doxorubicin, epirubicin, idarubicin, mitoxantrone and valrubicin. Typical cytoskeletal disruptors are paclitaxel, docetaxel, abraxane and taxotere. Typical histone deacetylase inhibitors are vorinostat and romidepsin. Typical topoisomerase I inhibitors are irinotecan and topotecan. Typical topoisomerase II inhibitors are etoposide, teniposide and tafluposide. Typical kinase inhibitors are bortezomib, erlotinib, gefitinib, imatinib, vemurafenib and vismodegib. Typical nucleotide analogs are azathioprine, capecitabine, cytarabine, doxifluridine, fluorouracil, gemcitabine, hydroxyurea, mercaptopurine, methotrexate and tioguanine. Typical retinoids are tretinoin, alitretinoin and bexarotene. Typical vinca alkaloids are vinblastine, vincristine and vindesine.

### A.4. The Encapsulation Method

The capsules according to the present invention are prepared using a ring-opening polymerization method, more preferably using interfacial ring opening polymerization. The interfacial ring opening polymerization is preferably an oil/solvent in water methodology, which is preferred, as it does not require full evaporation followed by redispersing in water. Another advantage of this methodology is that the compound to be encapsulated can be oil/solvent soluble. Hence the method allows to encapsulate hydrophobic compounds in a single step.

The ring opening polymerization of N-carboxy-anhydrides has been reviewed by Cheng and Deming (Top. Curr. Chem., 310, 1-26 (2012)). Primary and optionally secondary amines are the most obvious initiators and are widely used to initiate the ring opening polymerization via nucleophilic initiation. Basic initiators can initiate the ring opening polymerization via an activated monomer mechanism, starting by deprotonation of the NCA's followed by ring opening polymerization. When amine initiators are used, both mechanisms often run in parallel. Transition metal initiation is known to give better control on the polymerization. The use of hexamethyldisilazane as initiator has also been disclosed for better controlling the polymerization.

In a further preferred embodiment, a mixture of N-carboxy-anhydrides, derived from different amino acids is used. In an even further embodiment, a mixture of different chirality is used, preferably in a 9/1 to 1/9 ratio of a mixture of D- and L-amino acids. In another preferred embodiment, a mixture of chirality and different amino acids are used. Mixing D- and L-amino acids prevents the poly (amino acid) to form a secondary or tertiary structure as peptides do in nature. The obtained polymeric shell is hence denser and mechanically more resistant.

In a particularly preferred interfacial ring opening polymerization method for the preparation of the capsules according to the present invention, the N-carboxy-anhydride monomers and the NIR absorber are dissolved in a water immiscible solvent and emulsified in an aqueous solution containing a polymerization initiator. Upon emulsifying and optionally removing said water immiscible solvent, the ring opening polymerization is initiated at the interface. Upon propagation, a poly (amino acid) shell is formed at the organic-water interface, generating a core-shell structure, encapsulating the NIR absorber. The obtained polymeric shell is mechanically strong and stable and allows the capsule to be isolated from the liquid wherein the capsules have been prepared.

The particle size of the capsules of the invention is modified by modifying the emulsification technology, the use of an emulsification aid and the ratio of an emulsification aid to the shell and core during emulsification, the nature of the emulsification aid, changing the viscosity of the continuous or dispersed phase, the ratio of the continuous and dispersed phase, the nature of NIR absorber and the nature of the shell monomers. High shear technologies and ultrasound based technologies are particularly preferred as emulsification technologies. The particle size of the capsules according to the present invention can be tuned by tuning the shear in high shear technologies or by changing the power and amplitude upon sonification.

Di- or multifunctional primary or secondary amines or mixtures thereof are particularly preferred initiators for the ring opening polymerization of the NCA's. The initiators are water soluble and can be functionalized with additional hydrophilic functional groups, preferably selected from the group consisting of a carboxylic acid or salt thereof, a sulfonic acid or salt thereof, a phosphonic acid or salt thereof, a phosphate ester or salt thereof, a sulfate ester or salt thereof, a poly-hydroxyl functionalized group, a poly(ethylene glycol), an ammonium group, a sulfonium group and a phosphonium group.

Typical initiators are given in table 3 without being limited thereto

TABLE 3

| | |
|---|---|
| 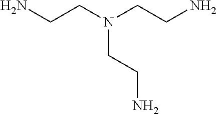 | INI-1 |
| 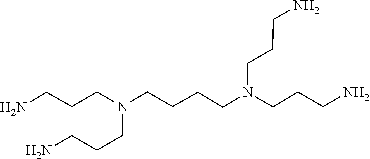 [SMILES: NCCCN(CCCN)CCCCN(CCCN)CCCN] | INI-2 |
| 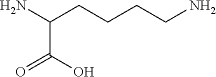 | INI-3 |
| 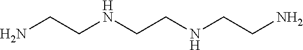 | INI-4 |
| 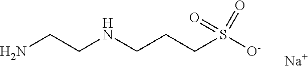 [SMILES: NCCNCCCS(=O)(=O)[O-] ; [Na+]] | INI-5 |
| 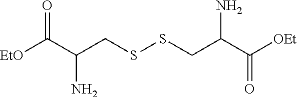 [SMILES: CCOC(=O)C(N)CSSCC(N)C(=O)OCC] | INI-6 |
| 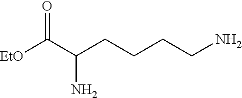 [SMILES: CCOC(=O)C(N)CCCCN] | INI-7 |
| 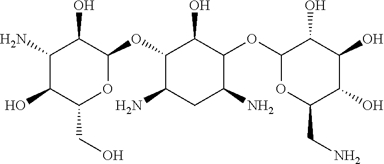 [SMILES: NC[C@H]1OC(OC2[C@@H](N)C[C@@H](N)[C@H](O[C@H]3O[C@H](CO)[C@@H](O)[C@H](N)[C@H]3O)[C@H]2O)[C@H](O)[C@@H](O)[C@@H]1O] | INI-8 |
| 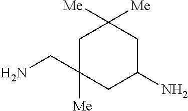 [SMILES: CC1(C)CC(N)CC(C)(CN)C1] | INI-9 |
| 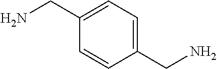 | INI-10 |
| 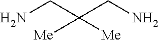 | INI-11 |
| 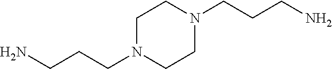 [SMILES: NCCCN1CCN(CCCN)CC1] | INI-12 |
| 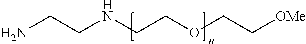 | INI-13 |
| n = 11 on average | |
| 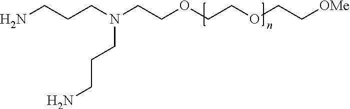 | INI-14 |
| n = 7 on average | |

The incorporation of a poly(ethylene gycol) functional group is particularly useful to give stealth properties to the capsules of the invention if used in the human or animal body. These stealth properties are required to avoid uptake by the reticuloendothelial system.

The poly(ethylene glycol) can be introduced using different strategies, including making use of poly(ethylene glycol) functionalized initiators, dispersion aids and poly(ethylene glycol) functionalized NCA monomers or combinations thereof. In a particularly preferred embodiment, a monomer according to general structure I is used, wherein at least one of R₁ to R₃ is functionalized with a poly(ethylene glycol) chain. In a particularly preferred embodiment, said monomer is selected from the group consisting of a cysteine derivative, a lysine derivative, an ornithine derivative, a glutamic acid derivative and an aspartic acid derivative. Typical examples of ethoxylated N-carboxy anhydrides are given in Table 4 without being limited thereto.

TABLE 4

| | |
|---|---|
| 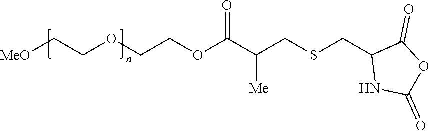 | PEG-NCA-1 |
| 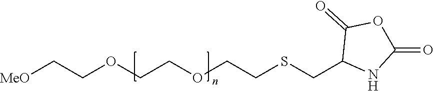 | PEG-NCA-2 |
| 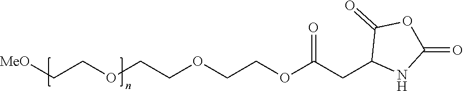 | PEG-NCA-3 |
| 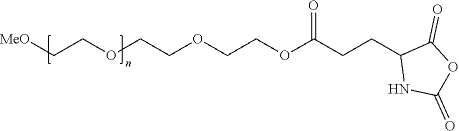 | PEG-NCA-4 |
| 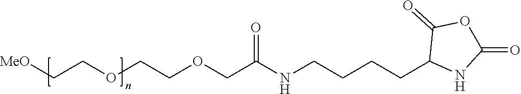 | PEG-NCA-5 |
| 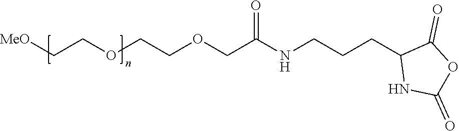 | PEG-NCA-6 |
| 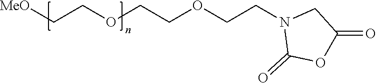 | PEG-NCA-7 |

The monomer content of ethoxylated N-carboxy anhydrides is preferably between 5 and 50 wt. % of the total monomer composition, more preferably between 10 and 40 wt. %.

In a further preferred embodiment, the capsule of the invention further comprises a crosslinker. After biocompatibility and biodegradability, one of the most basic requirements of the resin particle is stability in the medium wherein it has to function or has to be stored, e.g. the human body for non-invasive therapy or diagnostics. Increased stability results in increased storage stability and in an increased blood circulation time and increased bioavailability. With a crosslinker, the stability and mechanical resistance of the resin particle can be modified to meet the specifications of the system in which the resin particle is used.

Any crosslinker known to crosslink amine functionalized polymers can be used. Preferred crosslinkers are selected from the group consisting of di- or multifunctional isocyanates, di- or multifunctional β-keto-esters, di- or multifunctional β-keto-amides, di- or multifunctional 1,3-diketones, di- or multifunctional epoxides or oxetanes, di- or multifunctional anhydrydes, d- or multifunctional N-carboxy-anhydrides, di- or multifunctional Michael acceptors such as acrylates, methacrylates, maleimides, vinyl sulfones and the like and di- or multifunctional five membered carbonates.

Preferably, an additional emulsification aid is used during the emulsification step. Typical emulsification aids are selected from stabilizing polymers and surfactants. The polymers and surfactants can be co-reactive polymers or surfactants, e.g. functionalized with primary and secondary amines, taking the role of both initiator and emulsification aid, leading to so called self-dispersing capsules. The surfactant can be anionic, non-ionic, cationic or zwitterionic. As stabilizing polymers, hydroxyl functionalized polymers are particularly preferred, preferably selected from polysaccharides and poly(vinyl alcohol) or poly(vinyl alcohol) copolymers or derivatives thereof. Poly- or oligo(ethylene oxide) functionalized block- or star-copolymers are another class of particularly preferred polymeric emulsification aids.

In a further preferred embodiment, the NIR responsive capsule according to the present invention is a core-shell particle, comprising an additional functional compound in the core, capable of being released upon NIR laser exposure. The encapsulation technology according to the present invention is particularly of interest for the encapsulation of active pharmaceutical ingredients.

A particularly preferred interfacial ring opening polymerization method comprises the steps of
a) dissolving a compound according to general structure I and an organic NIR absorber in a water immiscible solvent; and
b) dissolving a polymerization initiator in an aqueous liquid; and
c) emulsifying the solution obtained in step a) into the aqueous liquid forming a solvent in water emulsion; and
d) optionally evaporating the water immiscible solvent; and
e) polymerizing the compound according to general structure I

If an additional functional compound such as a pharmaceutical active agent has to be integrated in capsule of the invention, this compound is preferably dissolved in the water immiscible solvent. The ring opening interfacial polymerization method according to the present invention is particularly of interest for the incorporation of active pharmaceutical ingredients such as anti-cancer drugs.

A particularly preferred method for the preparation of a dispersion of the composite resin particles according to the present invention, comprising an additional functional compound such as an active pharmaceutical ingredient includes the following steps:
a) dissolving a compound according to general structure I, an organic NIR absorber and an additional functional compound to be encapsulated in a water immiscible solvent; and
b) dissolving a polymerization initiator in an aqueous liquid; and
c) emulsifying the solution obtained in step a) into the aqueous liquid; and
d) optionally evaporating the water immiscible solvent; and
e) polymerizing the compound according to general structure I

### B. Fields of Application

The composite resin particles according to the invention are suitable for imaging affected organs in the human and/or animal body. The strong absorption of NIR light makes them also suitable for diffuse optical tomography and photoacoustic imaging.

When irradiated with an appropriate NIR laser, the NIR absorber in the composite particle of the invention can convert the absorbed photon energy into heat, directly ablating cancer cells with minimal invasion to surrounding healthy tissues making the particles very suitable in tumour phototherapy treatment (PTT).

The composite resin particle of the invention is also useful in photodynamic therapy (PDT) wherein the NIR absorber is excited with light of an appropriate wavelength for converting molecular oxygen into cytotoxic reactive oxygen species (ROS), such as singlet oxygen, which in turn damages cancer cells through oxidative stress and consequently induces cell death.

### C. Examples

### C.1. Materials

Mowiol 4 88 is a poly(vinyl alcohol) supplied by Kuraray.

Marlon A365 is an anionic surfactant supplied by Sasol Germany GMBH.

Tris(2-aminoethyl)amine was supplied by TCI.

Crosslinker-1 is a trifunctional β-keto-ester according to the following structure, which can be prepared as disclosed by Speisschaert et al. (Polymer, 172, 239-246 (2019)).

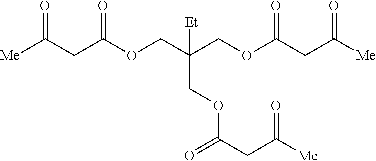 [SMILES: CCC(COC(=O)CC(C)=O)(COC(=O)CC(C)=O)COC(=O)CC(C)=O]

NIR-27 has a structure as given below and has been supplied by FEW as S2025

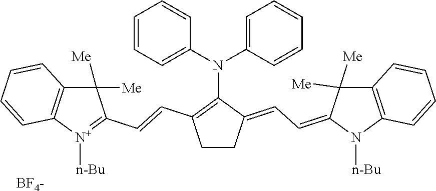

NIR-7 is a NIR absorber and is prepared as follows:

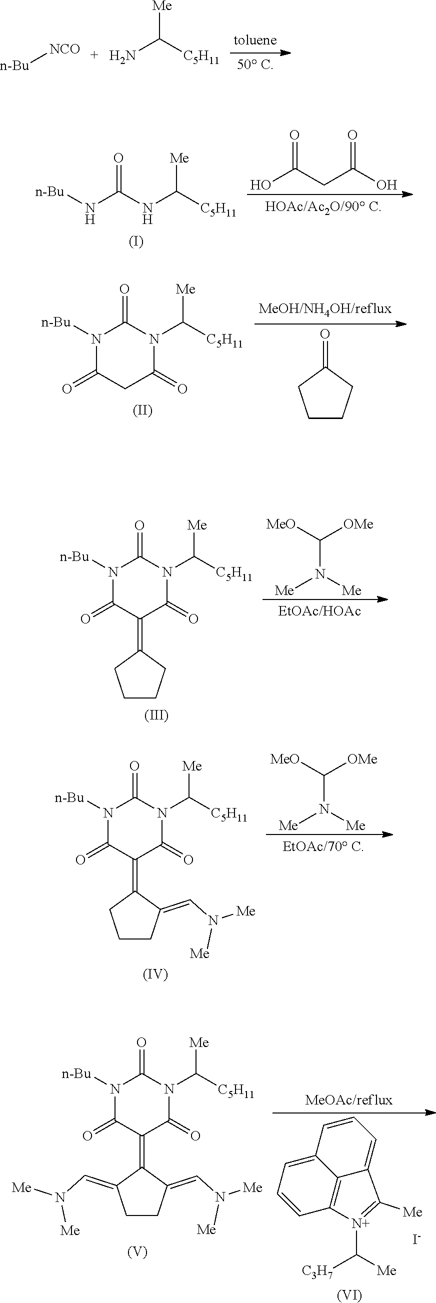

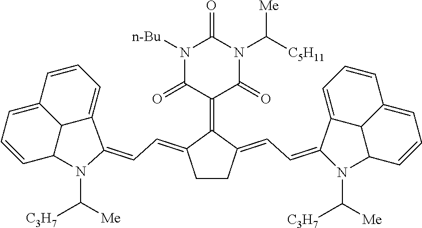

### The Synthesis of the Ureum (I)

146 g n.-butyl isocyanate was dissolved in 85 ml toluene. 166 g 2-heptyl amine was added over two hours while the temperature was kept below 50° C. The reaction was allowed to continue for 30 minutes at 50° C. The solvent and the excess of n.-butyl isocyanate were removed under reduced pressure and the crude ureum was used in the second step without further purification.

### The Synthesis of the Barbiturate (II)

206 g acetic acid was added to 300 g of the ureum (I). The mixture was heated to 60° C. The solution of ureum (I) in acetic acid was added to 147 g malonic acid at 60° C. This solution was added to 292 g acetic anhydride. The reaction mixture was gently heated to 90° C. and the reaction was allowed to continue for two and a half hours at 90° C. The reaction was allowed to cool down to 50° C. and 78 g methanol was added. The mixture was refluxed for 45 minutes. The mixture was allowed to cool down to room temperature and the solvent was evaporated under reduced pressure. The residue was redissolved in 311 g methyl t.butyl ether and extracted three times with 2010 g of a 5 w % sodium chloride solution. The solvent was removed under reduced pressure. Four times 35 ml toluene was added followed by removal under reduced pressure. The crude barbituric acid derivative (II) was used without further purification.

### The Synthesis of Intermediate (III)

84 g cyclopentanone was added to 240 g (0.85 mol) of the barbituric acid derivative (II). 5 g ammonium acetate was added followed by the addition of 101 g methanol. The reaction mixture was heated to reflux and the reaction was allowed to continue for four and a half hours at reflux. The reaction mixture was allowed to cool down to room temperature and the solvent was removed at 50 mbar pressure and 95° C. Four times 5 ml toluene was added followed by evaporation at 50 mbar and 100° C. The reaction mixture was allowed to cool down to room temperature and 92 g toluene was added. 26 g silicagel in 55 g toluene was added and the mixture was filtered. The silicagel was flushed with toluene. The pooled toluene fractions were treated twice with 26 g silicagel in 55 g toluene, filtered and followed by flushing the silicagel with toluene. All toluene fractions were pooled, followed by evaporation of the solvent under reduced pressure. 287 g (y: 97%) of the crude intermediate (III) was isolated.

### The Synthesis of Intermediate (V):

0.685 kg of intermediate (III) was dissolved in 0.334 kg ethyl acetate. The solution was cooled to 10° C. and 18.9 g acetic acid was added. 0.273 kg N,N-dimethylformamide dimethyl acetal was added over 10 minutes, while the temperature rose to 20° C. The reaction was allowed to continue for 30 minutes at room temperature. The reaction mixture was heated to 45° C. and 0.604 kg dimethylformamide dimethyl acetal was added over 15 minutes, followed by heating the reaction mixture to 65° C. The reaction was allowed to continue for 25 minutes at 65° C. The reaction mixture was allowed to cool to 47° C., followed by the addition of 1.06 kg methyl t.-butyl ether and 1.61 kg n.-hexane. The reaction mixture was cooled to 7° C. The crystallized intermediate V was isolated by filtration, washed with 160 g ethyl acetate and 60 g methyl t.-butyl ether, followed by three times washing with 160 g ethyl acetate and 60 g n.-hexane and once with 400 g heptane. The isolated intermediate (V) was dried. 335 g (y: 37%) of intermediate (V) was isolated.

### The Synthesis of Intermediate (VI):

Intermediate (VI) can be prepared as disclosed in WO2013037672.

### The Synthesis of NIR-7:

621 g of intermediate (VI) was dissolved in 3.2 I methyl acetate. The reaction mixture was heated to 40° C. 372 g of intermediate (V) was added and the reaction was allowed to continue for two and a half hours at 50° C. The reaction mixture is cooled to 20° C. The crystallized NIR-7 was isolated by filtration, washed with 284 ml methyl acetate, 2.84 I ethyl acetate and 530 ml methyl t.-butyl ether. The crude NIR-7 was treated in 2.9 L water, isolated by filtration, washed with 1.5 I water, 142 ml methyl acetate, 280 ml ethyl acetate and 800 ml methyl t.-butyl ether and dried. 597 g (y: 87%) of NIR-7 was isolated.

NIR-24 is a NIR absorber and is prepared as follows:

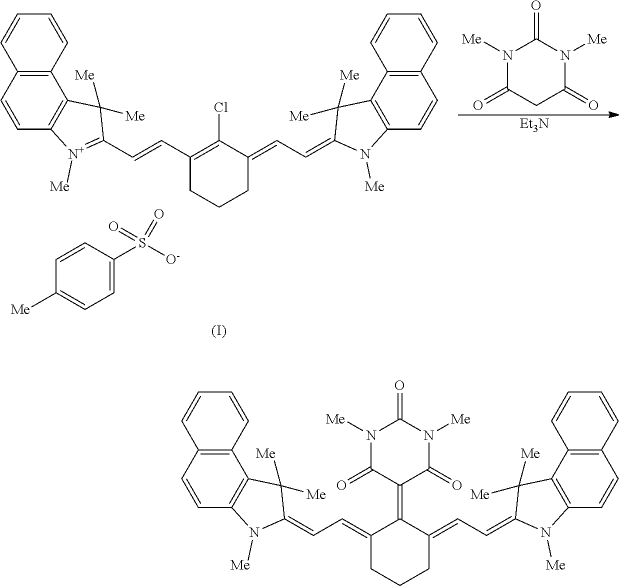 [SMILES: "Et3N" ; CN1/C(=C/C=C2\CCCC(/C=C/C3=[N+](C)c4ccc5ccccc5c4C3(C)C)=C2Cl)C(C)(C)c2c1ccc1ccccc21 ; CN1C(=O)CC(=O)N(C)C1=O ; Cc1ccc(S(=O)(=O)[O-])cc1 ; "(I)" ; CN1C(=O)C(=C2/C(=C/C=C3/N(C)c4ccc5ccccc5c4C3(C)C)CCC/C2=C\C=C2\N(C)c3ccc4ccccc4c3C2(C)C)C(=O)N(C)C1=O]

The starting NIR dye (I) can be prepared as disclosed by Nagao et al. (Dyes and Pigments, 73(3), 344-352 (2006).

### The Synthesis of NIR-24

30 g of NIR starting material (I) was added to 100 ml acetonitrile. 6.24 g N,N′-dimethyl barbituric acid was added, followed by the addition of 5.5 ml (4.0 g) triethyl amine. The reaction was allowed to continue for three hours at room temperature. The crude NIR-24 was isolated by filtration and treated in methanol at reflux. NIR-24 was isolated by filtration of the warm methanol solution and dried. 19.7 g (y: 35%) of NIR-24 was isolated.

NIR-25 is a NIR absorber and is prepared as follows:

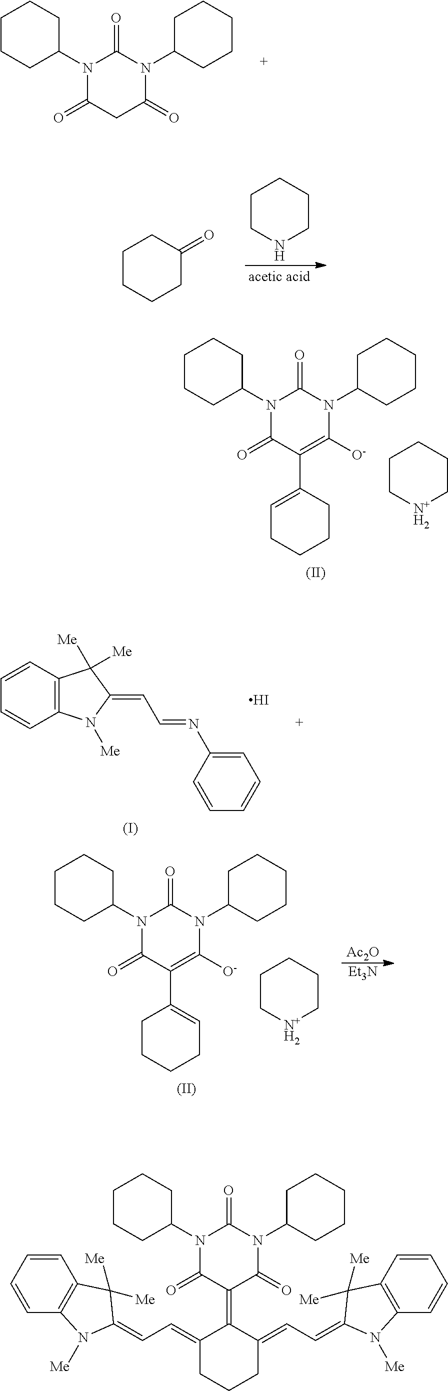

### The Synthesis of Intermediate (I):

Intermediate (I) can be prepared as disclosed in WO2010120058.

### The Synthesis of Intermediate (II):

292 g N,N′-dicyclohexyl barbituric acid was dissolved in 1.5 liter trichloroethane. 156 ml cyclohexanone, 12 ml piperidine and 15 ml acetic acid were added and the reaction mixture was heated to reflux. Water was removed by azeotrpic distillation using a Dean-Stark trap. The reaction was allowed to continue for 24 hours. The reaction mixture was allowed to cool down to room temperature and the solvent was removed under reduced pressure. The residue was redissolved in 1 liter trichloroethane. The undissolved residues were removed by filtration. 100 ml piperidine was added and intermediate (II) crystallized from the medium. Intermediate (II) was isolated by filtration and dried. 302 g (y: 67%) was isolated.

### The Synthesis of NIR-25:

301.6 g of intermediate (II) and 533 g of intermediate (I) were dissolved in 1.05 I of N,N′-dimethyl imidazolone. 186 ml acetic anhydride and 366 ml triethyl amine were added and the reaction mixture was heated to 100° C. The reaction was allowed to continue for 30 minutes at 100° C. The reaction mixture was allowed to cool down to room temperature and NIR-25 was allowed to crystallize from the reaction mixture. The crude NIR-25 was isolated by filtration and treated with 1.3 I acetone followed by treating with 1.3 I methyl t.butyl ether. The isolated NIR-25 was redissolved in a 1/1 mixture of dichloromethane and methanol. Residual impurities were removed by filtration. 6.6 I methyl t.butyl ether was added and NIR-25 was allowed to crystallize from the medium. NIR-25 was isolated by filtration and dried. 136 g (y: 28%) of NIR-25 was isolated.

NIR-26 is a NIR absorber and is prepared as follows:

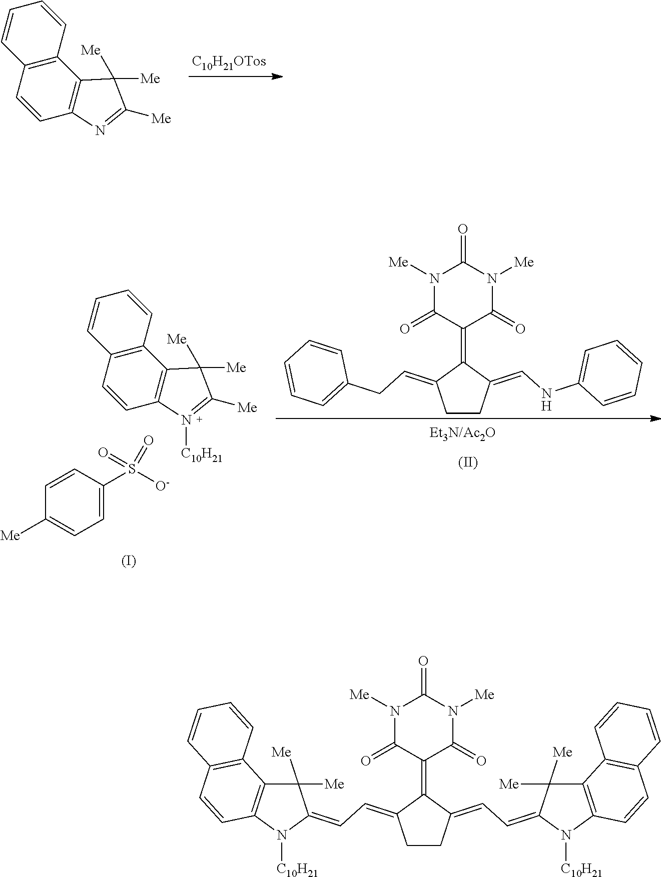

### The Synthesis of Intermediate (I):

123.2 g 1,1,2-trimethyl-1H-benzoindole and 234.1 g n-decyl tosylate were dissolved in 173 g sulfolane. A nitrogen flow was set over the reactor and the reaction mixture was heated to 123° C. The reaction was allowed to continue for 6 hours at 123° C. The reactor was cooled to 75° C. and 1350 ml ethyl acetate was added while stirring to crystallize intermediate (1). The reaction mixture was allowed to cool down to room temperature and the crystallized intermediate (I) was isolated by filtration. 207 (y: 69%) was isolated.

### The Synthesis of Intermediate (II):

Intermediate (II) can be prepared as disclosed in EP889363.

### The Synthesis of NIR-26:

2.14 g of intermediate (II) was dissolved in 10.9 ml acetic anhydride at 40° C. 2.27 g triethyl amine was added at 40° C. After 15 minutes, 1 ml dimethyl acetamide was added at 55° C. A solution of 5.2 g of intermediate (I) in 20 ml methanol was added at 55° C. The reaction was allowed to continue at 55° C. for three hours. The crystallized NIR-26 was isolated by filtration, washed with methanol, followed by treatment with methanol at 50° C. and isolation by filtration. NIR-26 was dried under reduced pressure at 40° C. 3 g (y: 69%) of NIR-26 was isolated.

NIR-11 is a NIR absorber and is prepared as follows:

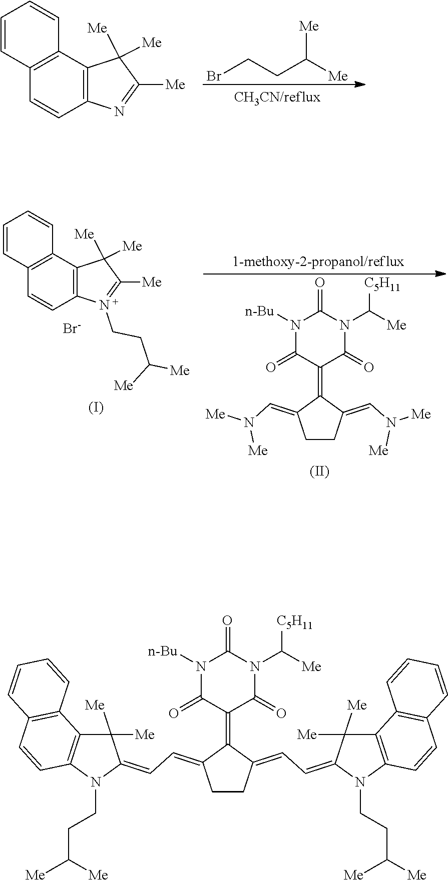

The Alkylation of 1,1,2-trimethyl-1H-benzoindole

31.4 g 1,1,2-trimethyl-1H-benzoindole and 33.0 g 1-bromo-3-methyl-butane were dissolved in 60 ml acetonitrile. The reaction mixture was heated to reflux and the reaction was allowed to continue for 20 hours at reflux. The reaction mixture was allowed to cool down and 20 ml acetonitrile was added. 100 ml methyl t.-butyl ether was added and the precipitated intermediate (I) was isolated by filtration, washed with methyl t.-butyl ether and dried. 24.9 g (y: 46%) of intermediate (I) was isolated.

### The Synthesis of NIR-11

1 g of intermediate (I) and 0.636 g of intermediate (II) were dissolved in 15 ml 1-methoxy-2-propanol. The reaction mixture was heated to reflux and the reaction was allowed to continue at reflux for one hour. The reaction mixture was allowed to cool down to room temperature. NIR-11 crystallized from the medium. NIR-11 was isolated by filtration, washed with 1-methoxy-2-dowanol and dried. 0.775 g (y: 59%) of NIR-11 was isolated.

NIR-28 is a NIR absorber and is prepared as follows:

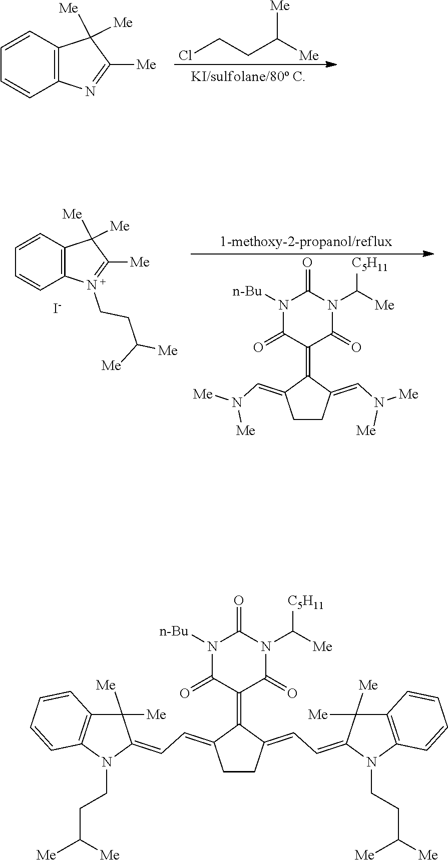

The Alkylation of 2,3,3-trimethyl-indolenine

32 g 2,3,3-trimethyl-indolenine and 25 g 1-chloro-3-methyl-butane were dissolved in 80 ml sulfolane. 40 g potassium iodide was added. The reaction mixture was heated to 80° C. and the reaction was allowed to continue for 19 hours at 80° C. The reaction mixture was allowed to cool down to room temperature and 30 ml acetone was added. The precipitated potassium chloride was removed by filtration and 600 ml ethyl acetate was added to the filtrate. Intermediate (I) crystallized from the medium. Intermediate (I) was isolated by filtration, washed with ethyl acetate and methyl t.-butyl ether and dried. 29.4 g (y: 41%) of intermediate (I) was isolated.

### The Synthesis of NIR-28

1 g of intermediate (I) and 0.641 g of intermediate (II) were dissolved in 15 ml 1-methoxy-2-propanol. The reaction mixture was heated to reflux and the reaction was allowed to continue at reflux for one hour. The reaction mixture was allowed to cool down to room temperature. NIR-28 crystallized from the medium. NIR-28 was isolated by filtration, washed with 1-methoxy-2-dowanol and dried. 0.716 g (y: 62%) of NIR-28 was isolated.

L-phenylalanine N-carboxy anhydride, D-phenylalanine N-carboxy anhydride and D,L-phenylalanine N-carboxy anhydride are N-carboxy-anhydride monomers and can be prepared according to standard methods as disclosed by Gabashvill et al. (Journal of Physical Chemistry B, 111(38), 11105-11110 (2007)) and Otake et al. (Angewandte Chemie, International Edition, 57(35), 11389-11393 (2018)).

L-leucine N-carboxy anhydride, D-leucine N-carboxy anhydride and D,L-leucine N-carboxy anhydride are N-carboxy-anhydride monomers and can be prepared according to standard methods as disclosed by Baars et al. (Organic Process Research and Development, 7(4), 509-513 (2003)).

PEG-NCA-1 is a N-carboxy-anhydride monomer and is prepared as follows:

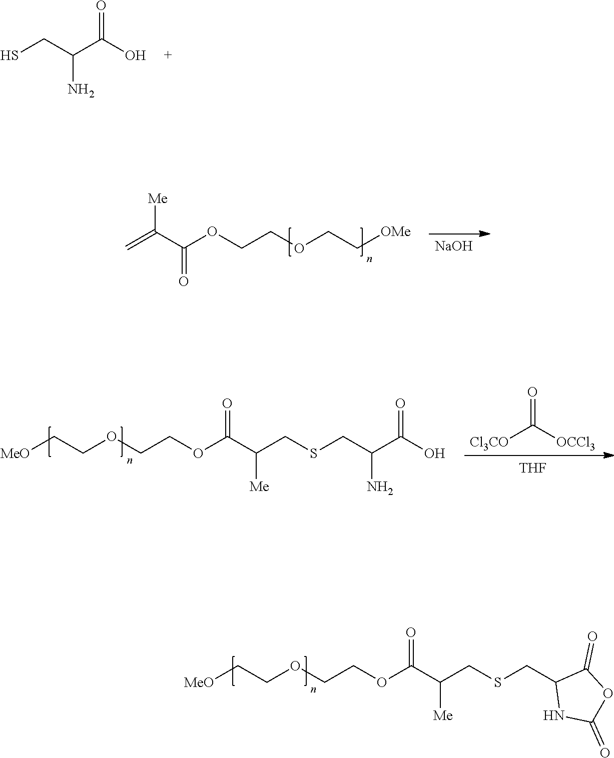

### The Addition of Cysteine to Pegylated Methacrylates

6.6 g cysteine was added to 75 ml water. The pH was adjusted to 7.5 using a 1 N NaOH solution. 23.6 g of methacrylated mono-methoxy-poly(ethylene glycol) 350 was added and the reaction was allowed to continue for 24 hours at room temperature. The salts in the aqueous solution were removed by using a chromatographic technique. The aqueous solution was pumped onto a Flashpure C18 (40 μm, irregular) column, supplied by Büchi. The column was flushed with water for several minutes, followed by elution of the cysteine derivative using methanol. The methanol fractions were evaporated under reduced pressure. The residue was dissolved in ethyl acetate. The ethyl acetate solution was dried over magnesium sulfate and the solvent was evaporated under reduced pressure. 28 g of the intermediate addition product of cysteine to methacrylated mono-methoxy-poly(ethylene glycol) 350 was isolated as a white wax.

### The Synthesis of PEG-NCA-1

100 ml tetrahydrofurane was added to 10 g of the pegylated cysteine derivative. 2.7 g trophosgen was added and the reaction was allowed to continue for three hours at 60° C. During the reaction, the pegylated cysteine derivative gradually dissolved. The reaction mixture was allowed to cool down to room temperature and the solvent was removed under reduced pressure. 50 ml n.-hexane was added and PEG-NCA-1 was isolated by decantation. The isolated PEG-NCA-1 was dissolved in 5 ml tetrahydrofurane, precipitated with 50 ml n.-hexane and isolated by decantation. This was repeated additionally three times. The isolated PEG-NCA-1 was dried under reduced pressure. 10 g (y: 96%) of PEG-NCA-1 was isolated as a viscous oil.

PEG-NCA-2 is a N-carboxy-anhydride monomer and is prepared as follows:

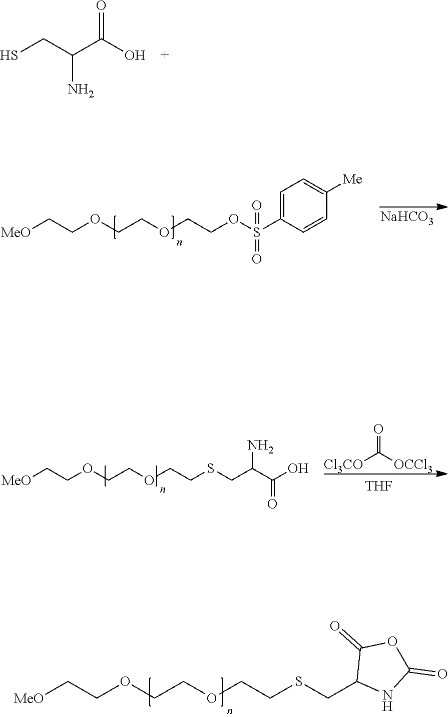

### The Alkylation of Cysteine:

6.06 g cysteine was added to 75 ml water. 16.8 g sodium bicarbonate was added followed by the addition of tosylated poly(ethylene glycol)monomethyl ether (prepared from poly(ethylene glycol)-monomethyl ether 550, using standard tosylation conditions as described by Cia et al (Macromolecules, 45(15), 6175-6184 (2012)). The reaction mixture was heated to 75° C. and the reaction was allowed to continue for six hours at 75° C. The reaction mixture was allowed to cool down to room temperature. The aqueous solution was pumped onto a Flashpure C18 (40 μm, irregular) column, supplied by Buchi. The column was flushed with water for several minutes, followed by elution of the cysteine derivative using methanol. The methanol fractions were evaporated under reduced pressure. The residue was dissolved in methylene chloride. The methylene chloride solution was dried over magnesium sulfate and the solvent was evaporated under reduced pressure. 22 g (y: 100%) of the pegylated cysteine derivative was isolated as a slightly colored wax.

### The Synthesis of PEG-NCA-2:

100 ml tetrahydrofurane was added to 10 g (15 mmol) of the pegylated cysteine derivative. 2.22 g (7.5 mmol) triphosgene was added and the mixture was heated to 60° C. The reaction was allowed to continue for three hours at 60° C. The pegylated cysteine derivative gradually dissolved upon reaction. The reaction was allowed to cool down to room temperature and the solvent was removed under reduced pressure. 100 ml n.-hexane was added and PEG-NCA-2 was isolated by decantation. The isolated PEG-NCA-1 was dissolved in 10 ml tetrahydrofurane, precipitated with 100 ml n.-hexane and isolated by decantation. This was repeated additionally twice. The isolated PEG-NCA-2 was dried under reduced pressure. 10 g (y: 95%) of PEG-NCA-2 was isolated as a viscous oil.

### C.2. Methods

The particle size of the capsules was measured using a Zetasizer™ Nano-S (Malvern Instruments, Goffin Meyvis).

The UV-VIS spectra were measured on an Agilent 8433 spectrophotometer for spectra up to 1100 nm. The more bathochromic dyes were measured on a Shimadzu UV2600 spectrophotometer. The absorption of the samples at Amax was adjusted to 1 by diluting the samples with water.

### C.3. Example 1

This example illustrates the encapsulation of different NIR absorbers by poly(amino acid) resins, using an anionic surfactant and a polymeric emulsification aid as stabilizing system.

### The Synthesis of INVRES-1

A first solution was prepared by dissolving 0.75 g L-phenylalanine N-carboxy anhydride, 0.75 g D-phenylalanine N-carboxy anhydride, 0.75 L-leucine N-carboxy anhydride, 0.75 g D-leucine N-carboxy-anhydride and 0.336 g crosslinker-1 in 20 ml methyl ethyl ketone. A solution of 75 mg NIR-7 in 1 ml dichloromethane was added to this solution. The solution was filtered over a 2.7 μm filter.

A second solution was prepared by dissolving 0.692 g Mowiol 4 88, 0.259 g Marlon A365 and 0.127 g tris(2-aminoethyl)amine in 30 ml water.

The first solution was added to the second solution using mixing with an Ultra Turrax T25 (IKA) at 15000 rpm for 5 minutes while maintaining the temperature of the emulsion between 2° and 30° C. 10 ml water was added followed by evaporation of the mixture under reduced pressure to 30 g. The polymerization was allowed to continue at room temperature for 24 hours.

The measured average particle size was 253 nm. The dispersion had an absorption maximum at 1051 nm.

### The Synthesis of INVRES-2

A first solution was prepared by dissolving 0.75 g L-phenylalanine N-carboxy anhydride, 0.75 g D-phenylalanine N-carboxy anhydride, 0.75 L-leucine N-carboxy anhydride, 0.75 g D-leucine N-carboxy-anhydride, 0.336 g crosslinker-1 and 75 mg NIR-26 in 26 ml dichloromethane. The solution was filtered over a 2.7 μm filter.

A second solution was prepared by dissolving 0.692 g Mowiol 4 88, 0.259 g Marlon A365 and 0.127 g tris(2-aminoethyl)amine in 30 ml water.

The first solution was added to the second solution using mixing with an Ultra Turrax T25 (IKA) at 18000 rpm for 5 minutes while maintaining the temperature of the emulsion between 2° and 30° C. 10 ml water was added followed by evaporation of the mixture under reduced pressure to 30 g. The polymerization was allowed to continue at room temperature for 24 hours.

The measured average particle size was 305 nm. The dispersion had an absorption maximum at 841 nm.

### The Synthesis of INVRES-3

A first solution was prepared by dissolving 1.5 g D,L-phenylalanine N-carboxy anhydride, 0.75 L-leucine N-carboxy anhydride, 0.75 g D-leucine N-carboxy-anhydride, 0.336 g crosslinker-1 and 75 mg NIR-24 in 26 ml dichloromethane. The solution was filtered over a 2.7 μm filter.

A second solution was prepared by dissolving 0.692 g Mowiol 4 88, 0.259 g Marlon A365 and 0.127 g tris(2-aminoethyl)amine in 30 ml water.

The first solution was added to the second solution using mixing with an Ultra Turrax T25 (IKA) at 15000 rpm for 5 minutes while maintaining the temperature of the emulsion between 2° and 30° C. 10 ml water was added followed by evaporation of the mixture under reduced pressure to 30 g. The polymerization was allowed to continue at room temperature for 24 hours.

The measured average particle size was 314 nm. The dispersion had an absorption maximum at 812 nm.

### The Synthesis of INVRES-4

A first solution was prepared by dissolving 1.5 g D,L-phenylalanine N-carboxy anhydride, 0.75 L-leucine N-carboxy anhydride, 0.75 g D-leucine N-carboxy-anhydride, 0.336 g crosslinker-1 and 75 mg NIR-27 in 26 ml dichloromethane. The solution was filtered over a 2.7 μm filter.

A second solution was prepared by dissolving 0.692 g Mowiol 4 88, 0.259 g Marlon A365 and 0.127 g tris(2-aminoethyl)amine in 30 ml water.

The first solution was added to the second solution using mixing with an Ultra Turrax T25 (IKA) at 15000 rpm for 5 minutes while maintaining the temperature of the emulsion between 2° and 30° C. 10 ml water was added followed by evaporation of the mixture under reduced pressure to 30 g. The polymerization was allowed to continue at room temperature for 24 hours.

The measured average particle size was 282 nm. The dispersion had an absorption maximum at 827 nm.

### The Synthesis of INVRES-5

A first solution was prepared by dissolving 1.5 g D,L-phenylalanine N-carboxy anhydride, 0.75 L-leucine N-carboxy anhydride, 0.75 g D-leucine N-carboxy-anhydride, 0.336 g crosslinker-1 and 75 mg NIR-25 in 26 ml dichloromethane. The solution was filtered over a 2.7 μm filter.

A second solution was prepared by dissolving 0.692 g Mowiol 4 88, 0.259 g Marlon A365 and 0.127 g tris(2-aminoethyl)amine in 30 ml water.

The first solution was added to the second solution using mixing with an Ultra Turrax T25 (IKA) at 15000 rpm for 5 minutes while maintaining the temperature of the emulsion between 2° and 30° C. 10 ml water was added followed by evaporation of the mixture under reduced pressure to 30 g. The polymerization was allowed to continue at room temperature for 24 hours.

The measured average particle size was 280 nm. The dispersion had an absorption maximum at 773 nm.

### The Synthesis of INVRES-6

A first solution was prepared by dissolving 1.5 g D,L-phenylalanine N-carboxy anhydride, 0.75 L-leucine N-carboxy anhydride, 0.75 g D-leucine N-carboxy-anhydride, 0.336 g crosslinker-1 and 75 mg NIR-11 in 26 ml dichloromethane. The solution was filtered over a 2.7 μm filter.

A second solution was prepared by dissolving 0.692 g Mowiol 4 88, 0.259 g Marlon A365 and 0.127 g tris(2-aminoethyl)amine in 30 ml water.

The first solution was added to the second solution using mixing with an Ultra Turrax T25 (IKA) at 15000 rpm for 5 minutes while maintaining the temperature of the emulsion between 2° and 30° C. 10 ml water was added followed by evaporation of the mixture under reduced pressure to 30 g. The polymerization was allowed to continue at room temperature for 24 hours.

The measured average particle size was 330 nm. The dispersion had an absorption maximum at 838 nm.

### The Synthesis of INVRES-7

A first solution was prepared by dissolving 1.5 g D,L-phenylalanine N-carboxy anhydride, 0.75 L-leucine N-carboxy anhydride, 0.75 g D-leucine N-carboxy-anhydride, 0.336 g crosslinker-1 and 75 mg NIR-28 in 26 ml dichloromethane. The solution was filtered over a 2.7 μm filter.

A second solution was prepared by dissolving 0.692 g Mowiol 4 88, 0.259 g Marlon A365 and 0.127 g tris(2-aminoethyl)amine in 30 ml water.

The first solution was added to the second solution using mixing with an Ultra Turrax T25 (IKA) at 15000 rpm for 5 minutes while maintaining the temperature of the emulsion between 2° and 30° C. 10 ml water was added followed by evaporation of the mixture under reduced pressure to 30 g. The polymerization was allowed to continue at room temperature for 24 hours.

The measured average particle size was 310 nm. The dispersion had an absorption maximum at 803 nm.

### C.4. Example 2

This example illustrates the synthesis of NIR responsive submicron particles, functionalized with poly(ethylene glycol) on the surface of the submicron particles.

### The Synthesis of INVRES-8:

A first solution was made by dissolving 1.5 g D,L-phenylalanine N-carboxy anhydride, 0.75 g L-leucine N-carboxy anhydride, 0.75 g D-leucine N-carboxy anhydride, 1.380 g PEG-NCA-1, 0.336 g crosslinker-1 and 121 mg NIR-7 in 14 ml ethyl acetate. The solution was filtered over a 2.7 μm filter.

A second solution was prepared by dissolving 0.968 g Synperonic PE and 0.127 g tris(2-aminoethyl)amine in 29 ml water.

The first solution was added to the second solution using mixing with an Ultra Turrax T25 (IKA) at 14000 rpm for 5 minutes while maintaining the temperature of the emulsion between 2° and 30° C. 10 ml water was added followed by evaporation of the mixture under reduced pressure to 30 g. The polymerization was allowed to continue at room temperature for 24 hours.

The measured average particle size was 151 nm. The dispersion had an absorption maximum at 1051 nm.

### The Synthesis of INVRES-9:

A first solution was made by dissolving 1.5 g D,L-phenylalanine N-carboxy anhydride, 0.75 g L-leucine N-carboxy anhydride, 0.75 g D-leucine N-carboxy anhydride, 1.380 g PEG-NCA-2, 0.336 g crosslinker-1 and 121 mg NIR-7 in 14 ml ethyl acetate. The solution was filtered over a 2.7 μm filter.

A second solution was prepared by dissolving 0.968 g Synperonic PE and 0.127 g tris(2-aminoethyl)amine in 29 ml water.

The first solution was added to the second solution using mixing with an Ultra Turrax T25 (IKA) at 14000 rpm for 5 minutes while maintaining the temperature of the emulsion between 2° and 30° C. 10 ml water was added followed by evaporation of the mixture under reduced pressure to 30 g. The polymerization was allowed to continue at room temperature for 24 hours.

The measured average particle size was 162 nm. The dispersion had an absorption maximum at 1051 nm.

### C.5. Example 3

This example illustrates the NIR response of dispersion comprising NIR responsive submicron particles according to the present invention.

A 1 mm thick coating of diluted dispersions of NIR nanoparticles according to the present invention was were exposed to laser radiation on a Coherent laser combination, equipped with 3 lasers, respectively emitting at 920 nm, 1064 nm and 1150 nm. It was evaluated to which extend the different 1 mm thick coating evaporated under laser exposure. The samples were coated on Priplak, a poly(propylene) substrate supplied by Antalis. The results are summarized in Table 5.

TABLE 5

| Resin particle | Dilution | Laser exposure | |
|---|---|---|---|
| dispersion | ratio | wavelength | Laser response |
| INVRES-8 | 1/5 | 1064 nm | Immediate full evaporation |
| | | | over the full power sweep |
| INVRE9-9 | 1/5 | 1064 nm | Immediate full evaporation |
| | | | over the full power sweep |
| INVRE9-9 | 1/5 | 1150 nm | Immediate full evaporation |
| | | | over the full power sweep |
| INVRES-4 | 1/1 | 920 nm | Immediate full evaporation |
| | | | over the full power sweep |
| INVRES-1 | 1/5 | 1064 nm | Immediate full evaporation |
| | | | over the full power sweep |

From Table 5, it becomes apparent that the NIR responsive nanoparticles according to the present invention show high laser response at different wavelengths.

1.-15. (canceled)

16. A capsule comprising a polymeric shell surrounding a NIR absorber, the polymeric shell comprises a poly(amino acid) and is obtainable by interfacial polymerization of a N-carboxy-anhydride monomer according to general structure I: 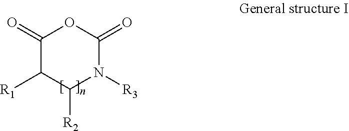
wherein
n represents 0 or 1
R₁, R₂ and R₃ are selected from the group consisting of a hydrogen, a substituted or unsubstituted alkyl group, a substituted or unsubstituted alkenyl group, a substituted or unsubstituted alkynyl group, a substituted or unsubstituted aralkyl group, a substituted or unsubstituted alkaryl group and a substituted or unsubstituted aryl or heteroaryl group
R₁, R₂ and R₃ may represent the necessary atoms to form a five to eight membered ring.

17. The capsule according to claim 16, wherein the shell comprises poly(ethylene glycol).

18. The capsule according to claim 16, wherein the NIR absorber is a compound according to general formula II: 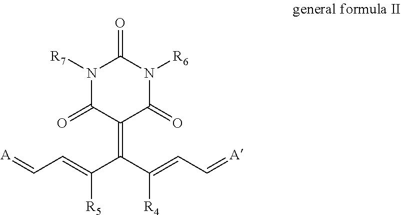
wherein
A and A′ independently represent a substituted or unsubstituted heterocyclic group, covalently bonded to the polymethine chromophore via a carbon atom
R₄ and R₅ are independently selected from the group consisting of a hydrogen, a substituted or unsubstituted alkyl group, a substituted or unsubstituted alkenyl group, a substituted or unsubstituted alkynyl group, a substituted or unsubstituted aralkyl group, a substituted or unsubstituted alkaryl group and a substituted or unsubstituted aryl or heteroaryl group
R₄ and R₅ may represent the necessary atoms to form a five to eight membered ring
R₆ and R₇ are independently selected from the group consisting of a substituted or unsubstituted alkyl group, a substituted or unsubstituted alkenyl group, a substituted or unsubstituted alkynyl group, a substituted or unsubstituted aralkyl group, a substituted or unsubstituted alkaryl group and a substituted or unsubstituted aryl or heteroaryl group.

19. The capsule according to claim 16, wherein the NIR absorber is a compound according to general formula III: 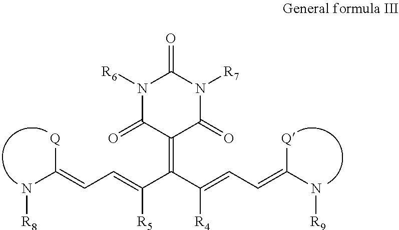
wherein,
R₄ and R₅ are independently selected from the group consisting of a hydrogen, a substituted or unsubstituted alkyl group, a substituted or unsubstituted alkenyl group, a substituted or unsubstituted alkynyl group, a substituted or unsubstituted aralkyl group, a substituted or unsubstituted alkaryl group and a substituted or unsubstituted aryl or heteroaryl group
R₄ and R₅ may represent the necessary atoms to form a five to eight membered ring
R₆ and R₇ are independently selected from the group consisting of a substituted or unsubstituted alkyl group, a substituted or unsubstituted alkenyl group, a substituted or unsubstituted alkynyl group, a substituted or unsubstituted aralkyl group, a substituted or unsubstituted alkaryl group and a substituted or unsubstituted aryl or heteroaryl group
R₈ and R₉ independently represent a substituted or unsubstituted alkyl group, a substituted or unsubstituted alkenyl group, a substituted or unsubstituted alkynyl group, a substituted or unsubstituted aralkyl group, a substituted or unsubstituted alkaryl group and a substituted or unsubstituted aryl or heteroaryl group
Q represents the necessary atoms to form a substituted or unsubstituted five or six membered heteroring.

20. The capsule according to claim 16, further having an average particle size from 0.07 μm to 5 μm.

21. The capsule according to claim 16, wherein the polymeric shell comprises a crosslinker.

22. The capsule according to claim 16, wherein the poly(amino acid) comprises an L-amino acid and a D-amino acid.

23. The capsule according to claim 16, wherein the interfacial polymerisation takes place in a solvent in water emulsion, the solvent being a water immiscible solvent and comprising the NIR absorber.

24. The capsule according to claim 16, wherein the polymeric shell surrounds a pharmaceutical active compound.

25. The capsule according to claim 18, wherein the polymeric shell surrounds a pharmaceutical active compound.

26. The capsule according to claim 19, wherein the polymeric shell surrounds a pharmaceutical active compound.

27. An aqueous dispersion comprising the capsules as defined in claim 16 and a surfactant or stabilizing polymer.

28. An aqueous dispersion comprising the capsules as defined in claim 18 and a surfactant or stabilizing polymer.

29. An aqueous dispersion comprising the capsules as defined in claim 19 and a surfactant or stabilizing polymer.

30. A pharmaceutical composition comprising the dispersion as defined in claim 27 and a pharmaceutical carrier or excipient.

31. A pharmaceutical composition comprising the dispersion as defined in claim 28 and a pharmaceutical carrier or excipient.

32. An aqueous dispersion as defined in claim 27 for use in medical imaging.

33. A method of preparing the dispersion as defined in claim 27, comprising the steps of:
a) dissolving a N-carboxy-anhydride monomer according to general structure I and a NIR absorber in a water immiscible solvent; and
b) dissolving a polymerization initiator in an aqueous liquid; and
c) emulsifying the solution obtained in step a) into the aqueous liquid; and
d) optionally evaporating the water immiscible solvent; and
e) polymerizing the N-carboxy-anhydride monomer according to general structure I.

34. The method of preparing the capsules according to claim 33, wherein a surfactant or hydrophilic polymer is added to the aqueous liquid.

35. The method of preparing the capsules according to claim 33, wherein the polymerization initiator is a di- or multifunctional primary or secondary amine comprising a polyethylene glycol group.
